(19) United States
(12) Patent Application Publication
(10) Pub. No.: US 2014/0123977 A1
(43) Pub. Date: May 8, 2014

(54) SLEEP-ACTIVATED CPAP MACHINE

(71) Applicant: Michael G. Lalonde, Alpharetta, GA (US)

(72) Inventor: Michael G. Lalonde, Alpharetta, GA (US)

(73) Assignee: DESHUM MEDICAL, LLC, Cambridge, MA (US)

(21) Appl. No.: 13/678,476

(22) Filed: Nov. 15, 2012

Related U.S. Application Data
(60) Provisional application No. 61/559,912, filed on Nov. 15, 2011.

(51) Int. Cl. A61M 16/00; A61B 5/021; A61B 5/0476; A61M 16/10; A61B 7/04; A61B 5/145; A61M 16/06; A61M 16/08; A61B 5/00; A61B 5/087

(52) U.S. Cl. CPC A61M 16/0051; A61M 16/0066; A61B 5/4836; A61B 5/4809; A61B 5/4812; A61B 5/021; A61B 5/0476; A61B 5/087; A61B 7/04; A61B 5/4818; A61B 5/14542; A61M 16/06; A61M 16/0816; A61M 16/1045; A61M 16/0875
USPC 128/201.13; 128/204.21; 128/204.23; 128/205.25

(57) ABSTRACT

A system and method for delaying the start of the continuous positive air pressure therein making it easier for a user to fall asleep. The system delivers pressurized gas to the airway of a patient. The system has a gas flow generator for providing a flow of gas and a mask for delivery of gas flow to an airway of a patient. The mask has an exhaust port being continuously open and having suitable flow resistance for maintaining a pressure in the cavity. The mask has a breathing port adaptable to open when no flow of pressurized air for allowing free breathing by the user. A hose extends between the gas flow generator and the mask for providing a flow of gas. The system has a mechanism for turning the flow of gas on at a time distinct from turning on the apparatus.

### CROSS-REFERENCE TO RELATED APPLICATIONS

This application claims priority under 35 U.S.C. §119 to the following U.S. Provisional patent applications, each of which is hereby incorporated by reference in its entirety: U.S. Ser. No. 61/559,912 filed Nov. 15, 2011.

### COPYRIGHT INFORMATION

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The present invention relates to a continuous positive airway pressure (CPAP) machine and more particularly to a CPAP machine that is activated based on the condition of the user.

### BACKGROUND OF THE INVENTION

The sleep apnea syndrome afflicts an estimated 1% to 5% of the general population and is due to episodic upper airway obstruction during sleep. Those afflicted with sleep apnea experience sleep fragmentation and intermittent, complete, or nearly complete cessation of ventilation during sleep with potentially severe degrees of oxyhemoglobin desaturation.

Although details of the pathogenesis of upper airway obstruction in sleep apnea patients have not been fully defined, it is generally accepted that the mechanism includes either anatomic or functional abnormalities of the upper airway which result in increased air flow resistance. Such abnormalities may include narrowing of the upper airway due to suction forces evolved during inspiration, the effect of gravity pulling the tongue back to oppose the pharyngeal wall, and/or insufficient muscle tone in the upper airway dilator muscles. It has also been hypothesized that a mechanism responsible for the known association between obesity and sleep apnea is excessive soft tissue in the anterior and lateral neck which applies sufficient pressure on internal structures to narrow the airway.

Recent work in the treatment of sleep apnea has included the use of continuous positive airway pressure (CPAP) to maintain the airway of the patient in a continuously open state during sleep. Unfortunately, the statistics on CPAP non-compliance are startling. There are numerous reasons for non-compliance including the discomfort of exhaling against a positive air pressure.

### SUMMARY OF THE INVENTION

It has been recognized that conventional CPAP (continuous positive airway pressure) machines to treat apnea provide a positive pressure to the user when the unit is turned on. The user is required to exhale, competing with the positive pressure from the flow generator. This competition against the CPAP machine is uncomfortable and not typical which results in difficulty falling asleep. It is recognized that by delaying the start of the continuous positive air pressure it is generally easier for a user to fall asleep.

In an embodiment of an apparatus for delivering pressurized gas to the airway of a patient, the apparatus includes gas flow generator, a mask, and a connector between the gas flow generator and the mask. The gas flow generator provides a flow of gas when the compressor is turned on. The mask is for delivering the flow of gas or pressured air to an airway of a patient. The apparatus has a mechanism for turning the flow of gas on at a time distinct from turning on the apparatus.

In an embodiment, the time distinct from turning on the apparatus is after the patient is asleep.

In an embodiment, the mechanism for turning the flow of gas on after the patient is asleep is a timer. In an embodiment, the timer is capable of being adjusted by a user. In an embodiment, the timer has a default time resulting in the delivery begins of pressurized gas flow to an airway of a patient a specific time after the unit turned on.

In an embodiment, the mechanism for turning the flow of gas on includes a mechanism to detect the sleep stage and turning on the flow of gas after the person is asleep.

In an embodiment, the mechanism to detect the sleep stage monitors the heart rate of the user. In an embodiment, the mechanism to detect the sleep stage monitors the brain waves of the user.

In an embodiment, the mechanism to detect the sleep stage monitors the breathing of the user. In an embodiment, the breathing is measured by monitoring the noise of breathing. In an embodiment, the breathing is measured by monitoring the change of pressure at the mask. In an embodiment, the breathing is measured by monitoring chest expansion. In an embodiment, the mechanism to detect the sleep stage compares characteristics associated with a sleep state as compared to a waking state in at least one of the body physiology of heart rate, brain waves, and breathing.

In an embodiment, the mechanism for turning the flow of gas on includes a mechanism to detect the onset of obstructive sleep apnea (OSA).

In an embodiment, the mechanism to detect OSA monitors the O₂ saturation of the user. In an embodiment, the mechanism to detect OSA monitors the air flow rate of the user. In an embodiment, the mechanism to detect OSA monitors the heart rate of the user. In an embodiment, the mechanism to detect OSA monitors the chest expansion of the user. In an embodiment, the mechanism to detect OSA monitors the blood pressure of the user.

In an embodiment of a mask for delivery of gas flow to an airway of a patient, the mask has a shell including a rim defining a cavity adapted for interface with a user's nose and mouth. The shell has a connection aperture. The mask has a mask connector which interfaces with the connection aperture of the shell. The connector defines a conduit for the flow of pressurized air from a flow generator. The mask has an exhaust port being continuously open and having suitable flow resistance for maintaining a pressure in the cavity. The mask has a breathing port adaptable to open when there is no flow of pressurized air for allowing free breathing by the user.

In an embodiment, the mask has a heat moisture exchange (HME) carried by the mask connector. The HME collects moisture on exhaling and provides moisture to the air on inhaling.

In an embodiment, the mask has a port defining a confined space. The port is adapted to connect a sensor carried on the flow generator. A flexible membrane, a button, covers the port and is adapted to change the volume of the confined space therein influencing the sensor.

In an embodiment, the mask has a second port adapted to connect to a sensor carried by the flow generator unit for controlling the air flow from the flow generator unit to the mask.

In an embodiment of a system for delivering pressurized gas to the airway of a patient, the system has a gas flow generator for providing a flow of gas and a mask for delivery of gas flow to an airway of a patient. The mask has a shell including a rim defining a cavity adapted for interface with a user's nose and mouth. The shell has a connection aperture. The mask has a mask connector interfacing with the connection aperture of the shell. The connector defines a conduit for the flow of pressurized air from the flow generator. The mask has an exhaust port being continuously open and having suitable flow resistance for maintain a pressure in the cavity. The mask has a breathing port adaptable to open when there is no flow of pressurized air to allow for free breathing by the user. A hose extends between the gas flow generator and the mask for providing a flow of gas. The system has a mechanism for turning the flow of gas on at a time distinct from turning on the apparatus.

In an embodiment, the system has an orientation sensor to determine the orientation of the unit and adjust the function dependent on the orientation.

In an embodiment, the orientation sensor is located on the gas flow generator unit. In an embodiment, the orientation sensor is located on the mask.

These aspects of the invention are not meant to be exclusive and other features, aspects, and advantages of the present invention will be readily apparent to those of ordinary skill in the art when read in conjunction with the following description, appended claims, and accompanying drawings.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a schematic of a CPAP system with sleep activity control system;

FIG. 2 is a sectional view of a portion of the mask;

FIG. 3 is a schematic of the sleep-activated timer system;

FIG. 4 is a schematic of the timer;

FIG. 5 is a schematic of the sleep-activated system that detects if a person is asleep;

FIG. 6 is a schematic of a method for detecting sleep stage; and

FIG. 7 is a schematic of a method for detecting obstructive sleep apnea.

### DETAILED DESCRIPTION OF THE INVENTION

A system and method for delivering pressurized gas to the airway of a patient, the system has a gas flow generator for providing a flow of gas and a mask for the delivery of the gas flow to an airway of a patient. The mask has a shell including a rim defining a cavity adapted for interface with a user's nose and mouth. The shell has a connection aperture. The mask has a mask connector interfacing with the connection aperture of the shell. The connector defines a conduit for the flow of pressurized air from the flow generator. The mask has an exhaust port being continuously open and having suitable flow resistance for maintaining a pressure in the cavity. The mask has a breathing port adaptable to open when there is no flow of pressurized air to allow for free breathing by the user. A hose extends between the gas flow generator and the mask for providing a flow of gas. The system has a mechanism for turning the flow of gas on at a time distinct from turning on the apparatus.

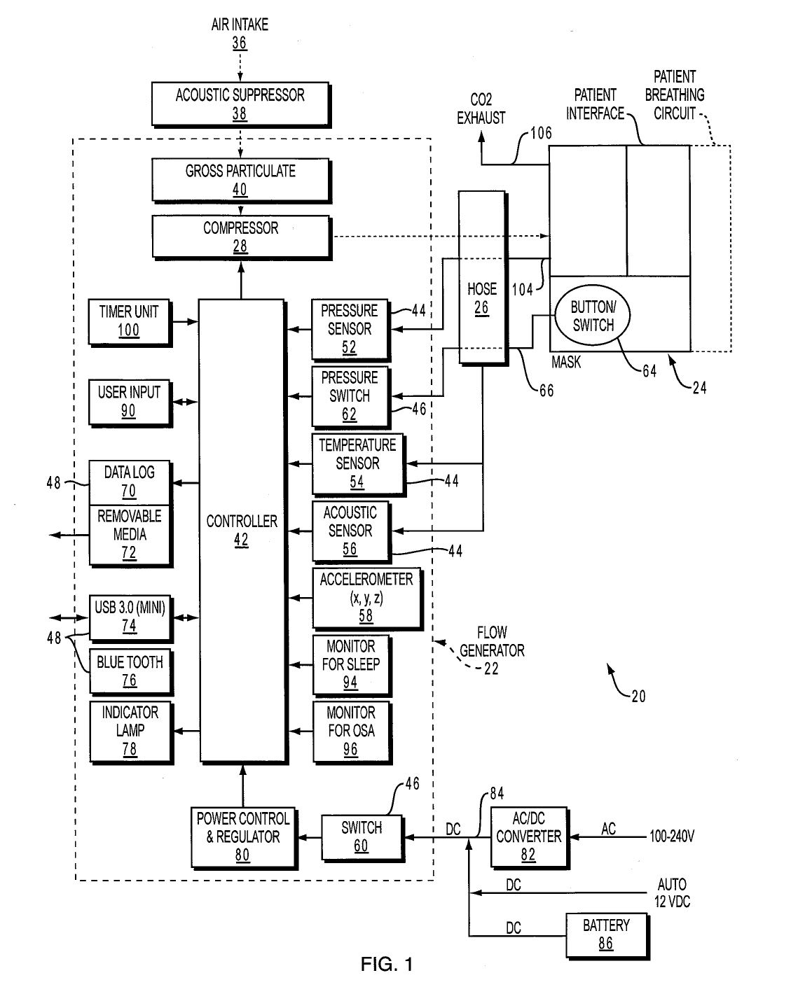

Referring to FIG. 1, a schematic of a CPAP system 20 with a sleep activity control system is shown. The system 20 has a blower unit 22, also referred to as a gas flow generator, for providing a source of pressurized breathable air, a patient interface 24, such as a mask, that is removably worn by the patient, and an interconnector 26, such as a hose. The blower unit 22 has a compressor 28 for taking ambient air and creating pressurized air flow. The pressure range desired can vary, but generally falls in the range of between 4 and 20 centimeters of water. The average user/patient however requires between 6 and 14 centimeter of water. Air flow from the compressor can be adjusted for a rate of 20 to 60 liters of air per minute.

The air for the mask 24 is drawn in at an air intake 36 and passes through an acoustic suppressor 38 and a filter 40 prior to the blades of the impeller of the compressor 28. The compressor 28 compresses the air increasing the pressure; an expansion chamber of the compressor allows the compressed air to expand and increase the velocity of the air. The pressurized air is passed through the interconnector 26 to the mask 24.

The blower unit 22 in addition has a controller 42 and a plurality of sensors 44, switches 46, and interface devices 48 for controlling the compressor 28.

The sensors 44 can include a pressure sensor 52 that monitors the pressure of the air in either the blower unit 22, the interconnector 26, and/or the mask 24. The sensors 44 can also include a temperature sensor 54, an acoustic sensor 56, and an accelerometer 58. The plurality of switches 46 includes a switch 60 for the system 20 located on the blower unit 22. In addition, the system 20 has a pressure switch 62 which connects to a switch 64 on the mask 24 with a conduit 66 carried by the interconnector 26.

The interface devices 48 include a data log 70 associated with removable media 72. The interface devices 48 can also include a USB port 74, blue tooth 76, and an indicator lamp 78.

Still referring to FIG. 1, the blower unit 22 has a power control and regulator 80 interposed between the switch 60 and the controller 42. The system 20 can be powered by various methods as represented by the AC/DC converter 82, a DC output 84 such as from an auto, and/or a battery 86. While 12 volts DC is shown, it recognized that the system may receive power inputs at a different voltage such as 14-15 volts, 19 volts, or 24 volts.

The system 20 has a user input 90 that allows the user/clinician to select/modify the working of the system 20. For example, the clinician can adjust the pressures or mode of treatment. The mode could include mono-level CPAP, Bi-level CPAP, and ramping. The user can select for example when the blower turns on as described in the paragraph below.

In addition, the blower (flow generator) unit 22 has a timer unit 100 that is capable of controlling when the compressor 28 is on and providing pressured air to the patient interface, mask 24 through the interconnector 26. In addition, the blower unit 22 in certain embodiments has an interface device 94 for detecting and monitoring sleep stages; as explained in more detail below, the interface device takes input from a sensor and determines if the user is asleep. In addition in certain embodiments, the blower unit 22 has a second or alternative interface device 96 for monitoring for detecting obstructed sleep apnea.

The mask 22 is most commonly a nasal mask or a full face mask as shown. It is recognized that the patient interface 22 can be other devices such as a nasal cannulae, an endotracheal tube, or any other interface, as explained below, based on other suitable appliances for interfacing between a source of breathing gas and a patient.

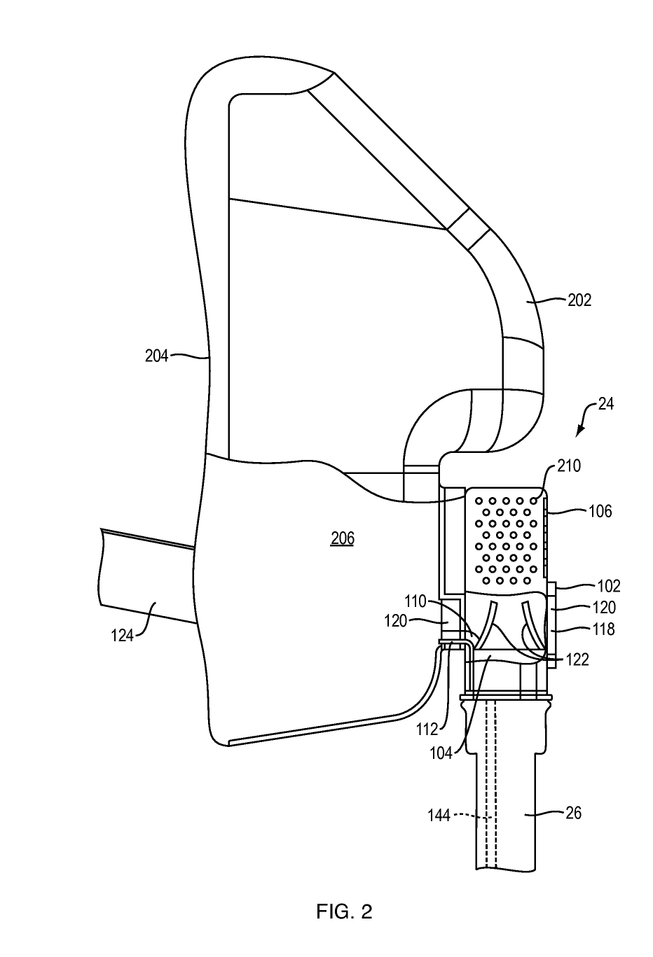

Referring to FIG. 2, a sectional view of a portion of the mask 24 is shown. The mask 24 has an inlet 104 for receiving pressurized gas from the blower unit 22. In addition, the mask 24 has a suitable exhaust port 106, schematically indicated in FIG. 1, and shown in FIG. 2 for exhaust of breathing gases during expiration. The inlet 104 and exhaust port 106 are part of a mask connector 102 that has a connector 108 that connects to a shell 202 of the mask 24. The mask 24 including the shell 202 has a rim 204 defining a cavity 206 adapted for interface with a user's nose and mouth. The shell 202 has a connection aperture 208. It is recognized that the mask 24 can have soft gel interface to the face.

Exhaust port 106 preferably is a continuously open port which imposes a suitable flow resistance upon exhaust gas flow to permit a pressure controller system 110 including a port 112 in the mask, a conduit 114, shown in hidden line, through the hose 26 to the pressure sensor 52 which through the controller 42, as seen in FIG. 1, which controls the pressure of air flow from the compressor 28 to the mask 24.

In one embodiment, the exhaust port 106 may be of sufficient cross-sectional flow area to sustain a continuous exhaust flow of approximately 15 liters per minute. The flow via the exhaust port 106 is one component, and typically the major component of the overall system leakage, which is an important parameter of system operation.

In addition still referring to FIG. 2, the mask 22 has a breathing port 118 that is open when the compressor 28 is not providing pressurized air at a specific rate. The breathing port 118 includes a plurality of the opening 120 and a pair of flaps 122 that covers the opening 120 when pressurized area is flowing from the hose 26 to the mask connector 102.

Still referring to FIG. 2, the system 20 has a heat moisture exchange (HME) component 210. The HME component 210 carried by the mask connector 102 collects moisture as the user exhales that passes through the exhaust port 106. As the user receives pressured air from the blower unit 22 through the mask connector 102, the pressurized air flows through the HME 210 to the cavity 206 with the shell 202 of the mask 24 therein providing moisture to the air.

Referring to FIG. 1, the timer 100 of the system 20 is capable of turning on the compressor at a different time than when the blower unit 22 or system 20 is turned on. The user input 90 allows the user to select the timer mode and particulars about the timer mode.

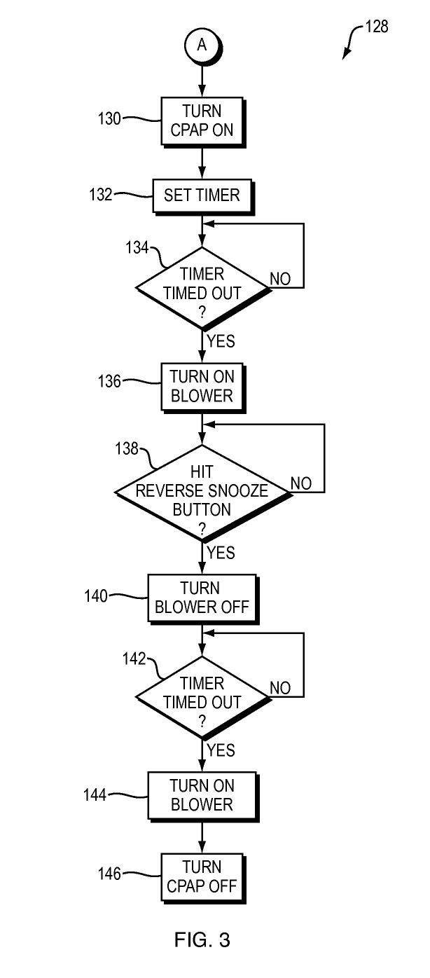

Referring to FIG. 3, a schematic of a sleep-activated timer system 128 is shown. A user turns on the system 20 as represented by block 130. Turning on the system 20 places the compressor 28 in a standby mode. In the embodiment shown, the user either enters a time into the timer or a default time is used as represented by block 132.

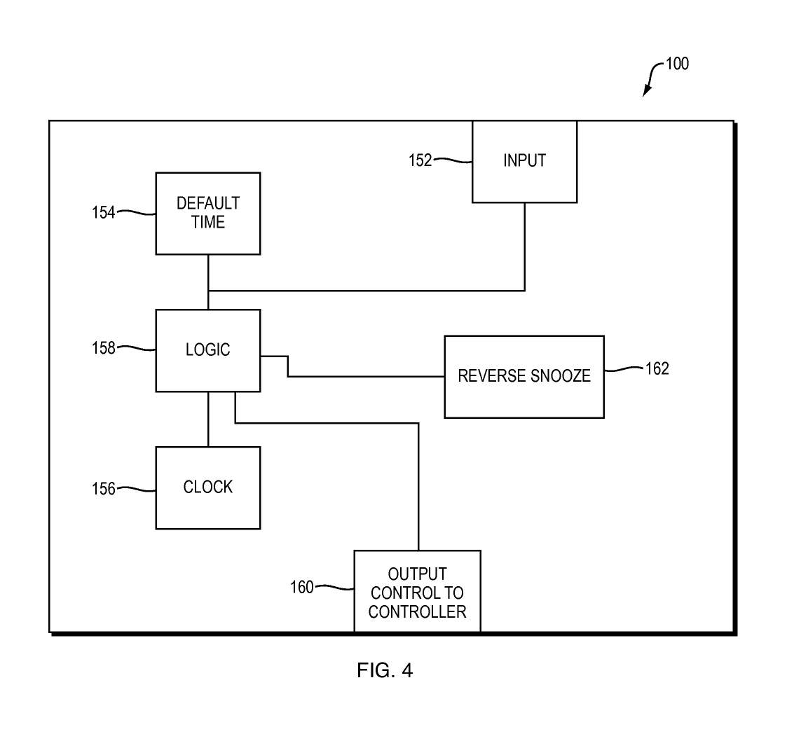

Referring to FIG. 4, a schematic of the timer 100 is shown. The input for the timer is represented by block 152 and the memory hold for the default time is represented by block 154. The timer has a clock as represented by block 156 and a logic circuit as represented by block 158. The timer unit 100 is fed to the controller via an output as by block 160.

Referring to back to FIG. 3, the system 128 continues to have the compressor 28 in a stand-by mode until the timer has timed out. If the timer has not timed out as represented by the “no” branch of decision diamond 134, the system 128 continues to monitor the timer. If the timer has timed out as represented by the “yes” branch of the decision diamond 134, the system turns on the compressor as represented by block 136. The system 20 can be a mono-level CPAP therapy, bi-level therapy, or other conventional therapy. In addition, the system can include a ramp function wherein after the compressor 28 is turned on, the pressure gradually increases until the system 20 reaches the prescribed pressure.

This allows the user to fall asleep without the discomfort of having to breathe, especially exhausting against the pressure of the CPAP machine.

In one embodiment the system has a “reverse snooze” button on the blower unit as represented by block 162 in FIG. 4. If a person wakes in the middle of night to a point of being sufficiently awake that the pressurized air is bothersome, the person could hit the “reverse snooze” button. If the user hits the reverse snooze button as represented by the “yes” branch from the decision diamond 138, the system turns the compressor 28 off as represented by block 140. It is contemplated that the user does not want to adjust the timer during their contemplated sleep cycle. The system can be set-up to allow the user to adjust the “reverse snooze” button to a percentage of the initial timer, a specific time, or a default time. It is generally recognized that a user will generally fall asleep more quickly during the middle of their contemplated sleep cycle than at the beginning of the cycle. The system 128 continues to monitor and when the timer times out as represented by the “yes” branch of the decision diamond 142, the blower is turned back on as represented by block 144. If the timer has not timed out as represented by the “no” branch of the decision diamond 142, the system 128 continues to monitor to determine when the timer times out.

Still referring to FIG. 3, when the user wakes up after the contemplated sleep cycle, the user can turn the system 20 off as represented by block 146.

Referring to FIG. 4, a schematic of the timer 100 is shown. The output from the controller 160 of the timer 100, which is connected to the controller 42, receives input from the logic circuit 158. The logic circuit 158 receives input from the user input 152, the default time 154, a clock 156, and the user input as reverse snooze input 162.

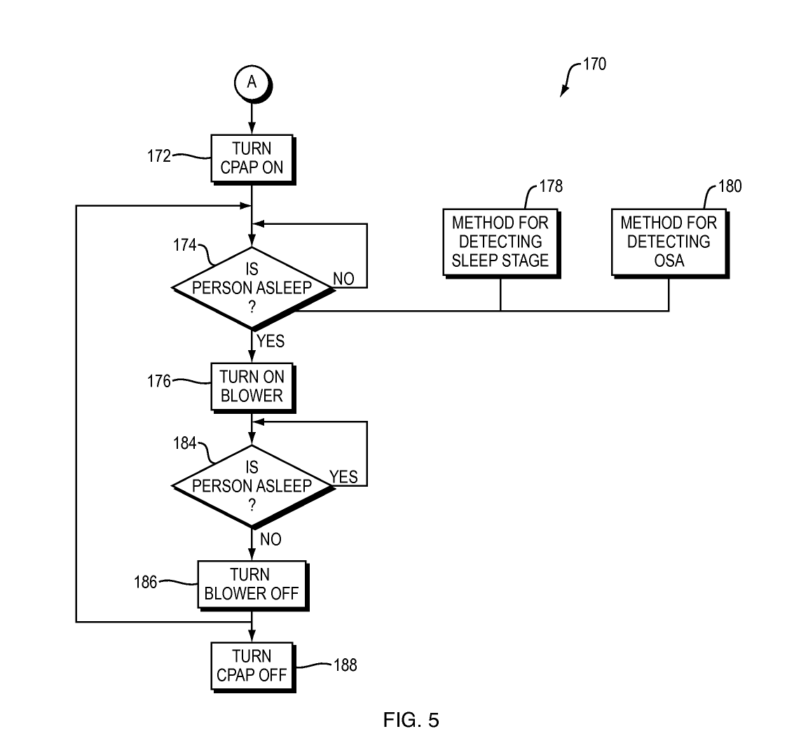

Referring to FIG. 5, a schematic of a sleep activation system 170 is shown. A user turns on the system 20, as represented by block 172. Turning on the system 20 places the compressor 28, seen in FIG. 1, in a standby mode. The user places the mask 24 on their face and goes to sleep breathing normally; referring back to FIG. 2, the user breathes using the breathing port 118, wherein the user is free breathing without pressure from the CPAP system.

The system 170 monitors to determine if the user is asleep as discussed in further detail below. If the system 170 determines that the person is still awake as represented by the “no” branch of decision diamond 174, the system 170 continues to monitor to determine if the user is still awake. If the system 170 determines the user is asleep as represented by the “yes” branch of the decision diamond 174, the system 170 turns on the compressor 28 as represented by block 176. The system 20 can be a mono-level CPAP therapy, bi-level therapy, or other conventional therapy. In addition, the system 20 can include a ramp function wherein after the compressor 28 is turned on, the pressure gradually increases until the system reaches the prescribed pressure. This allows the user to fall asleep without the discomfort of having to breathe, especially exhausting against the pressure of the CPAP machine.

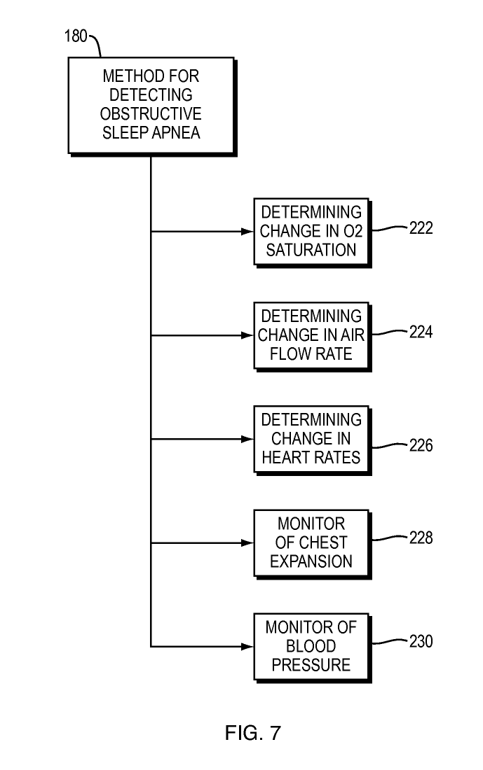

Still referring to FIG. 5, the method of determining if the user is asleep can be done by various methods. Block 178 represents determining sleep by detecting sleep stage. Block 180 represents determining if obstructed sleep apnea (OSA) is occurring. The method of determining both sleep stage and obstructed apnea (OSA) is discussed in further detail below. It is recognized that while the label “determining if the user is asleep” is used, that detection of OSA is not detecting sleep but rather apnea which is a sleep disorder characterized by abnormal pauses in breathing or instances of abnormally low breathing (i.e., reduced amplitude), during sleep; a person asleep without OSA could sleep the entire night without the blower (compressor) being turned on as explained in greater detail with respect to FIG. 7.

The system 170 continues to monitor to determine if the user continues to be asleep. If the person continues to sleep, the decision diamond 184 as represented by the “yes” branch, the system 170 continues to monitor. If a person wakes in the middle of night (or the OSA detected has gone away) the system determines that the user has woken up as represented by the “no” branch from the decision diamond 184. The system turns the blower off as represented by the block 186. The arrow from below the “turn blower off” block 186 to above the “Is person asleep?” decision diamond 174 represents that the system 170 will continue to monitor and turn the blower (Compressor) 28 on and off as necessary during the user's sleep cycle (or occurrence of OSA) until the user turns the system 20 off as represented by block 188.

While numbers are given as examples, it is recognized that what is normal for one person is not normal for another person. A professional is required to adjust the parameters for the particular user.

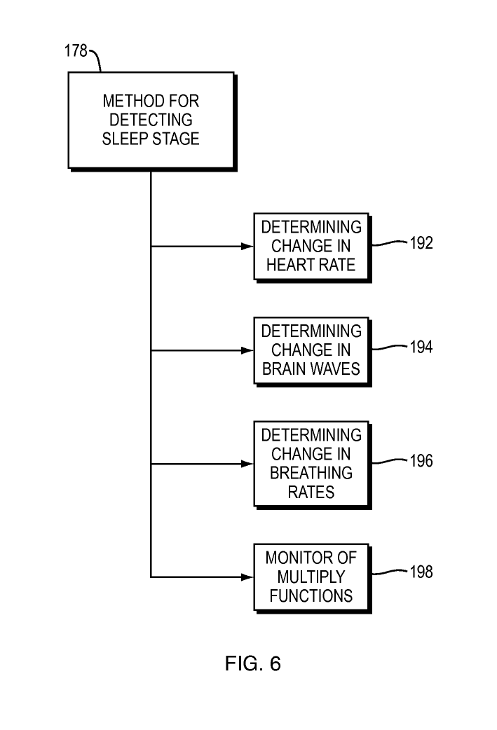

Referring to FIG. 6, a schematic of a method for detecting sleep stage is shown. As indicated above, the system 170 turns the blower on and off by monitoring and detecting when the user is in a sleep stage as represented by block 192. In one embodiment, the system 170 monitors the heart rate through a device such as a chest strap or finger pulse oximeter. The system 170 determines when the pulse drops below a certain level and turns on the compressor 28. The pulse rate at which the system transitions can be set by the clinician. It is recognized that a range would be selected so that the system would not cycle quickly. In one embodiment, the system 170 turns on the compressor 28 when the heart rate drops below 68 beats per minute. The system 170 turns off the compressor 28 when the heart rate rises above 72 beats per minute.

In an alternative arrangement, the system 170 begins to monitor the heart rate of the user as soon as the system is turned on. After taking a sufficient sample, the system determines the user's normal awake pulse rate. The system determines that the user is asleep by noticing a drop in pulse rate. The criteria for turning on the compressor can be either a drop in a certain percentage (e.g. a drop of 5 percent such as from 70 beats per minute to below 66.5 beaters per minute) or a drop of a certain number of beats per minute (e.g., a drop of 4 beats per minute regardless if the awake pulse is 60 beats per minute or 85 beats per minute.) Similar to the above embodiment, the system selects a range so it does not cycle.

In another embodiment, the system 170 monitors the brain waves of the user as represented by block 194 such as using a brain wave monitor having a plurality of electrodes mounted on the mask 24 or associated strap 124. The electrodes measure the potential change of the electric field generated by neurons in the brain. This procedure is generally referred to electroencephalography (EEG) and produces continuously varying sine waves. The waves are classified by their dominant frequency and represent different state of alertness. The system 170 monitors beta, alpha, theta, and delta characteristic brain waves.

The system begins to monitor the electrodes shortly after the system 20 is turned on. The system 170 determines changes in brain waves. When the system 170 determines that a user is asleep based on a change in the dominant brain wave, as represented by the decision diamond 174 in FIG. 5, the system 20 turns on the blower 28 as represented by block 176. The system 170 continues monitoring by the loop from decision diamond 174 to the turn blower off block 186 shown in FIG. 5 and described above.

In another embodiment, the system 170 monitors to determine if there is a change of breathing rate as represented by block 196 in FIG. 6. Changes in breathing rate are more than changes in frequency. During sleep the respiratory frequency remains about the same as during wakeful states at rest, but the tidal volume or amplitude of breathing is reduced. The system 170 could include a device in the mask 24 to measure airflow including amplitude, such as a pneumotachometer or spirometer.

In contrast to the heart rate monitoring or the brain wave monitoring, it may not be desirable to monitor breathing rate when using a nasal mask or nasal cannulae, in that a person's breathing rate may not be adequately monitored.

As with both the monitoring of heart rate and brain waves, the system 170 as seen in FIG. 5 will turn the blower (compressor) 28 on and off based on when the system 170 determines that the user is asleep.

Block 198 of FIG. 6 represents the capability of monitoring more than one trait of the user such as both heart rate and breathing rate to determine sleep status.

Referring to FIG. 7, a schematic of a method for detecting obstructive sleep apnea is shown. As indicated above, the system turns the blower on and off by monitoring and detecting when the user is having OSA. While the above methods link the activation of the compressor to sleeping, the following methods link the activation of the compressor to the user presenting signs related to OSA. OSA (obstructive sleep apnea) is a condition in which the flow of air pauses or decreases during breathing while you are asleep because the airway has become narrowed, blocked, or floppy. A pause in breathing is called an apnea episode. A decrease in airflow during breathing is called a hypopnea episode.

The purpose of the CPAP (continuous positive airway pressure) machine is to provide a positive pressure on the nasopharynx and the oropharynx to keep these airways open therein allowing the user to continue to breathe. In that the system is monitoring for detecting obstruction, the system is going to have more times when obstruction is not detected than when sleep is not detected. Therefore the user is able to free breath and not need to fight the air pressure from the unit. In addition, the system has a range such that turning the blower on and off occurs at different points such that the blower does not continue to cycle. Examples of ranges are discussed below; it is recognized that the actual range can be set by clinician.

In one embodiment, the system 170 monitors changes in O₂ saturation as represented by block 222. The system 170 has a pulse oximeter (saturometer). The pulse oximeter is a portable unit that has a pair of small light-emitting diodes (LEDs) facing a photodiode through a translucent part of the patient's body. The portable unit typically attaches to a fingertip or an earlobe, which are translucent parts of the patient's body. The unit has a red LED and an infrared LED. Absorption at these wavelengths differs significantly between oxyhemoglobin and its deoxygenated form; therefore, the oxy/deoxyhemoglobin ratio can be calculated from the ratio of the absorption of the red and infrared light. The absorbance of oxyhemoglobin and deoxyhemoglobin is the same (isosbestic point) for the wavelengths of 590 and 805 nm.

The pulse monitor can be located at various positions for example on a finger or on the ear lobe.

In one embodiment, the system monitors the change in air flow rate. Similar to the embodiment discussed above with respect to FIG. 6, the system 170 monitors to determine if there is a change of breathing rate as represented by block 224 in FIG. 7. One distinction that OSA has from normal breathing while sleeping is that the breathing rate varies as the user with sleep apnea stops breathing and then begins breathing again after waking up. The system 170 could include a device in the mask 24 to measure airflow including amplitude, such as a pneumotachometer or spirometer to measure respiratory frequency and the tidal volume or amplitude of breathing.

In another embodiment as represented by block 226 in FIG. 7, the system 170 determines when the pulse drops between a certain level and turns on the compressor. As indicated above with respect to a method for detecting sleep stage 178, the system 170 monitors the heart rate through a device such as a chest strap or a finger pulse oximeter.

In another embodiment as represented by block 228 in FIG. 7, the system monitors chest expansion to determine OSA. A strap having a series of strain gauges is worn by the user to measure the expansion.

In another embodiment as represented by block 230 in FIG. 7, the system monitors blood pressure. The system incorporates a wearable continuous monitoring blood pressure sensor such as developed at Massachusetts Institute of Technology by Dr. Harry Asada et al.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

What is claimed:

1. An apparatus for delivering pressurized gas to the airway of a patient, the apparatus comprising:
a gas flow generator for providing a flow of gas;
a mask for delivery of gas flow to an airway of a patient;
a connector between the gas flow generator and the mask for providing a flow of gas; and
a mechanism for turning the flow of gas on at a time distinct from turning on the apparatus.

2. An apparatus of claim 1 wherein the time distinct from turning on the apparatus is after the patient is asleep.

3. An apparatus of claim 2 wherein the mechanism for turning the flow of gas on after the patient is asleep is a timer.

4. An apparatus of claim 3 wherein the timer is capable of being adjusted by a user.

5. An apparatus of claim 3 wherein the timer has a default time resulting in the delivery begins of pressurized gas flow to an airway of a patient a specific time after the unit turned on.

6. An apparatus of claim 2 wherein the mechanism for turning the flow of gas on includes a mechanism to detect sleep stage and turns the flow of gas on after the person is sleeping.

7. An apparatus of claim 6 wherein the mechanism to detect sleep stage monitors the heart rate of the user.

8. An apparatus of claim 6 wherein the mechanism to detect sleep stage monitors the brain waves of the user.

9. An apparatus of claim 6 wherein the mechanism to detect sleep stage monitors the breathing of the user.

10. An apparatus of claim 9 wherein the breathing is measured by monitoring the noise of breathing.

11. An apparatus of claim 9 wherein the breathing is measured by monitoring the change of pressure at the mask

12. An apparatus of claim 9 wherein the breathing is measured by monitoring chest expansion.

13. An apparatus of claim 6 wherein the mechanism to detect sleep stage compares characteristics associated with a sleep state as compared to a wake state in at least one of heart rate, brain waves, and breathing.

14. An apparatus of claim 1 wherein the mechanism for turning the flow of gas on includes a mechanism to detect the onset of obstructive sleep apnea (OSA).

15. An apparatus of claim 14 wherein the mechanism to detect OSA monitors the O2 saturation of the user.

16. An apparatus of claim 14 wherein the mechanism to detect OSA monitors the air flow rate of the user.

17. An apparatus of claim 14 wherein the mechanism to detect OSA monitors the heart rate of the user.

18. An apparatus of claim 14 wherein the mechanism to detect OSA monitors the chest expansion of the user.

19. An apparatus of claim 14 wherein the mechanism to detect OSA monitors the blood pressure of the user.

20. A mask for delivery of gas flow to an airway of a patient comprising:
a shell having a rim defining a cavity adapted for interface with a user's nose and mouth, the shell having a connection aperture;
a mask connector interfacing with the connection aperture of the shell, the connector defining a conduit for flow of pressurized air from a flow generator;
an exhaust port being continuously open and having suitable flow resistance for maintaining a pressure in the cavity;
a breathing port adaptable to open when there is no flow of pressurized air for allowing free breathing by the user.

21. A mask of claim 20 further comprising a heat moisture exchange (HME) carried by the mask connector, the HME that collects moisture on exhaling and provides moisture to the air on inhaling.

22. A mask of claim 20 further comprising a port defining a confined space, the port adapted to connect a sensor carried on the flow generator, a flexible membrane covering the port adapted to change the volume of the confined space therein influencing the sensor.

23. A mask of claim 20 further comprising a port adapted to connect to a sensor carried by the flow generator unit for controlling the air flow.

24. A system for delivering pressurized gas to the airway of a patient, the system comprising:
a gas flow generator for providing a flow of gas;
a mask for delivery of gas flow to an airway of a patient, the mask having a shell including a rim defining a cavity adapted for interface with a user's nose and mouth, the shell having a connection aperture, the mask having a mask connector interfacing with the connection aperture of the shell, the connector defining a conduit for flow of pressurized air from the flow generator, the mask has an exhaust port being continuously open and having suitable flow resistance for maintain a pressure in the cavity, and the mask having a breathing port adaptable to open when no flow of pressurized air for allowing free breathing by the user;
a hose extending between the gas flow generator and the mask for providing a flow of gas; and
a mechanism for turning the flow of gas on at a time distinct from turning on the apparatus.

25. An apparatus of claim 24 wherein the time distinct from turning on the apparatus is after the patient is asleep.

26. A system of claim 24 wherein the mask has a port defining a confined space, the port adapted to connect a sensor carried on the flow generator, a flexible membrane covering the port adapted to change the volume of the confined space therein influencing the sensor.

27. A system of claim 24 further comprises an orientation sensor to determine the orientation of the unit and adjusting the function dependent on the orientation.

28. A system of claim 27 wherein the orientation sensor is located on the gas flow generator unit.

29. A system of claim 27 wherein the orientation sensor is located on the mask.
